(19) United States
(12) Patent Application Publication
(10) Pub. No.: US 2018/0344167 A1
(43) Pub. Date: Dec. 6, 2018

(54) X-Ray-Induced Acoustic Computed Tomography (XACT) Breast Imaging

(71) Applicant: The Board of Regents of the University of Oklahoma, Norman, OK (US)

(72) Inventors: Liangzhong Xiang, Norman, OK (US); Shanshan Tang, Norman, OK (US); Pratik Samant, Norman, OK (US)

(21) Appl. No.: 16/057,568

(22) Filed: Aug. 7, 2018

Related U.S. Application Data
(63) Continuation of application No. PCT/US2017/019344, filed on Feb. 24, 2017.
(60) Provisional application No. 62/300,124, filed on Feb. 26, 2016.

(51) Int. Cl. A61B 5/00; G06T 11/00; G06T 15/00

(52) U.S. Cl. CPC A61B 5/0093; A61B 5/4312; A61B 5/708; G06T 2207/10116; G06T 15/00; A61B 5/0095; G06T 2207/10136; G06T 11/003

(57) ABSTRACT

An X-ray computed tomography breast imaging method comprises positioning a patient such that a breast of the patient is disposed adjacent to an ultrasound detector comprising a transducer array, wherein the transducer array comprises a plurality of ultrasonic transducer elements, positioning an X-ray source in a predetermined position directed toward the breast, actuating the X-ray source to emit an X-ray pulse into the breast to induce ultrasonic acoustic waves to emit from the breast, wherein the X-ray pulse has a duration in a range of 1 picosecond (ps) to 1 microsecond (μs), detecting the ultrasonic acoustic waves with the ultrasound detector, and transmitting signals from the transducer array to a data processing system for generating an image of the breast.

### CROSS-REFERENCE TO RELATED APPLICATIONS

This application is a continuation application of international patent application number PCT/US2017/19344 filed on Feb. 24, 2017 by The Board of Regents of the University of Oklahoma and titled “X-Ray-Induced Acoustic Computed Tomography (XACT) Breast Imaging,” which claims priority to U.S. provisional patent application No. 62/300,124 filed on Feb. 26, 2016 by The Board of Regents of the University of Oklahoma and titled “X-ray Induced Acoustic Computed Tomography Breast Imaging,” which are incorporated by reference.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Breast cancer is the most frequently diagnosed malignancy for adult females and is second only to lung cancer in causing cancer-related deaths in the United States. The risk of radiation-induced cancer formation has recently become of particular concern in the medical imaging community due to the rapid increase in CT procedures being performed over the past several decades. The United States has gone from performing less than 5 million CT scans a year in 1980 to 75 million a year in 2010, with the number of CT scans performed each year increasing by approximately 10%. The prevailing theory regarding radiation dose and cancer incidence is that the risk of cancer incidence from exposure to low levels of ionizing radiation increases linearly with cumulative dose, and there is no threshold dose below which the magnitude of the risk is zero. The linear, no-threshold model is the driving motivation behind the concerted effort among clinicians, radiologists, researchers, government agencies, and manufacturers of radiologic imaging systems to reduce the radiation dose to patients as low as reasonably achievable.

Mammography is used for breast cancer screening throughout the world, and the recent reduction in breast cancer mortality is largely attributed to earlier detection. However, specificity and the positive predictive value of mammography remain limited owing to an overlap in the appearances of benign and malignant lesions, which is common for all types of projection imaging techniques. The sensitivity with which conventional mammography can identify malignant tumors in the preclinical phase will largely be affected by the nature of the surrounding breast parenchyma.

In the last decades, MR imaging of the breast has gained a role in clarifying determinate cases after mammography. However, long scanning times and the high cost of MR examinations have hampered the integration of MR into routine clinical practice. The constant trade-off between spatial and temporal resolution in MR has made it difficult to achieve the spatial resolution necessary for improved cancer detection and characterization. Ultrasonography is also widely used in clinical breast cancer detection, but it has poor resolution in characterizing lesion margins and identifying microcalcifications. Ultrasound is also extremely operator dependent. Other imaging approaches for breast cancer detection, including scintimammography, positron emission tomography, optical imaging, photoacoustic imaging, microwave imaging, and thermoacoustic imaging, have advantages and disadvantages of their own. But, generally no other modality has been able to compete with mammography in terms of detection performance, imaging time, and cost-effectiveness.

In recent years, dedicated breast CT has received intensive attention due to its wider accessibility (e.g., lower costs) than MRI, higher sensitivity than mammography, and lack of breast compression. For example, Koning Breast CT was recently approved by the FDA for breast cancer diagnosis. Dedicated breast CT imaging can provide for 3D lesion morphology, which can serve as a diagnostic indicator, and for better quantitative assessment of breast glandular content, a likely risk factor for breast cancer. However, there are known challenges such as visualization of microcalcifications due to image noise and loss of contrast, as well as cone-beam artifacts. More importantly, breast CT scanners can expose patients to cumulative radiation doses which may elevate individuals' lifetime risk of developing cancer. Alternative methods which provide improved imaging with lower doses of radiation would be desirable. It is to this goal that the embodiments of the present disclosure are directed.

### BRIEF DESCRIPTION OF THE DRAWINGS

Several embodiments of the present disclosure are illustrated in the appended drawings. It is to be noted however, that the appended drawings only illustrate several typical embodiments and are therefore not intended to be considered limiting of the scope of the present disclosure. The figures are not necessarily to scale and certain features and certain views of the figures may be shown as exaggerated in scale or in schematic in the interest of clarity and conciseness.

FIG. 1A is a schematic diagram of conventional breast CT.

FIG. 1B is a schematic diagram of breast XACT.

FIG. 2 shows a schematic diagram of a breast XACT imaging system in use.

FIGS. 3A-3C show side, top, and perspective views of an acoustic detector cup used in the XACT breast imaging system of the present disclosure.

FIG. 4 shows a side view of the positioning of an X-ray tube of the imaging system in relation to the acoustic detector.

FIG. 5 is a top view depicting an array of ultrasound transducer elements positioned on an inner shell of the acoustic detector cup.

FIG. 6 is a side view depicting dimensions for selecting X-ray energy levels according to breast size.

FIG. 7 is a schematic depicting two filters used for selecting and modifying the X-ray beam.

FIG. 8 is a schematic showing circuitry of an embodiment of the XACT breast imaging system of the present disclosure.

FIG. 9 is a schematic showing data flow in an embodiment of the XACT breast imaging system of the present disclosure.

FIG. 10 shows an alternative imaging table upon which a patient can lay during use of an embodiment of the XACT breast imaging system of the present disclosure.

FIG. 11 an alternative imaging model for use by standing patients during use of an embodiment of the XACT breast imaging system of the present disclosure.

FIG. 12 is a graph illustrating X-ray energy dependent X-ray penetration depth versus calcification-breast tissue imaging contrast.

FIG. 13 is a graph illustrating frequency-dependent ultrasound penetration depth versus spatial resolution.

FIG. 14 is an image of an observed conventional breast CT.

FIG. 15 is a segmentation of the observed breast CT in FIG. 14.

FIG. 16 is a simulated XACT image based on the observed conventional breast CT slice in FIG. 14.

FIG. 17 is an image of the ROI in FIG. 16.

FIG. 18 shows the signal intensity for the image of the ROI in FIG. 17 across the dotted line.

FIG. 19 is a flowchart illustrating a method of breast imaging according to an embodiment of the disclosure.

FIG. 20 is a flowchart illustrating a method of breast imaging according to another embodiment of the disclosure.

### DETAILED DESCRIPTION

It should be understood at the outset that, although an illustrative implementation of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

The following abbreviations and initialisms apply:

ADC: analog-to-digital conver(ter/sion)

cm: centimeter(s)

CT: computed tomography

dB: decibel(s)

FDA: Food and Drug Administration

K: Kelvin

keV: kiloelectron volt(s)

kVp: peak kilovolt(s)

MeV: megaelectron volt(s)

MHz: megahertz

mm: millimeter(s)

MR: magnetic resonance

mGy: milligray(s)

ms: millisecond(s)

mSv: milliSievert(s)

NEP: noise-equivalent pressure

PET: polyethylene terephthalate

ps: picosecond(s)

RF: radio frequency

ROI: region of interest

TDM: time-division multiplexing

XA: X-ray-induced acoustic

XACT: x-ray-induced, acoustic-computed tomography

μm: micrometer(s)

μs: microsecond(s)

2D: two-dimensional

3D: three-dimensional.

Before describing various embodiments of the present disclosure in more detail by way of exemplary description, examples, and results, it is to be understood that the present disclosure is not limited in application to the details of methods and compositions as set forth in the following description. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error. Further, in this detailed description, each numerical value (e.g., temperature or time) should be read once as modified by the term “about” (unless already expressly so modified), and then read again as not so modified unless otherwise indicated in context. As noted, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range. The use of the term “about” may mean a range including ±10% of the subsequent number unless otherwise stated.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

The present disclosure is directed to an XACT system and method for 3D breast imaging using X-ray-induced acoustic waves. In at least certain embodiments of this system and method, a single, non-rotated X-ray pulse is used to generate 3D ultrasound waves. The generated waves can be detected by a detector such as a linear transducer array, 2D transducer ring array, or 3D transducer array, and reconstructed as 2D or 3D breast images. A transducer is a device that converts one form of energy to another form of energy. In this context, the transducers convert XA waves to electrical signals. In at least one embodiment, the XACT system includes an X-ray tube and a hemispherical acoustic detector cup comprising an array of ultrasonic transducer elements positioned on an inner layer of the detector cup. A breast to be imaged is positioned within the acoustic detector cup. Unlike in conventional breast CT, rotation of the X-ray tube is unnecessary because a single exposure of X-ray pulse is generally sufficient to generate the ultrasonic waves which effect the transducer signals used to construct the breast image. The transducer signals from the detector arrays are sent to a multi-channel parallel data receiver for construction of a 3D tomographic image of the breast. The 3D reconstruction eliminates lesion overlap and provides a complete, true 3D description of the breast to determine whether the breast comprises an abnormality, such as a calcification. Meanwhile, the radiation dose is dramatically reduced as well. Due to the low intensity of the X-ray pulse required, which may operate with nanosecond or picosecond pulses, the imaging system of the present disclosure can operate at sub-mSv levels of radiation. Thus, the present system and method can reduce the radiation exposure to the patient by a factor of 10, even as compared with the newest FDA approved dedicated breast CT for breast cancer diagnosis, the Koning Breast CT, which the FDA approved on Jan. 14, 2015. The presently disclosed breast imaging system can also reduce the radiation dose, increase the imaging speed, and increase the imaging resolution. The imaging system is applicable to other radiation induced acoustic imaging modalities. In the method of the present disclosure, one X-ray pulse is sufficient to generate a breast imaging in 3D volume by using an ultrasound detector as described herein. One or more additional x-ray pulses can enhance image contrast, so more than one x-ray pulse can be delivered if such an enhanced image contrast is desired.

XACT Breast Imaging System and Method

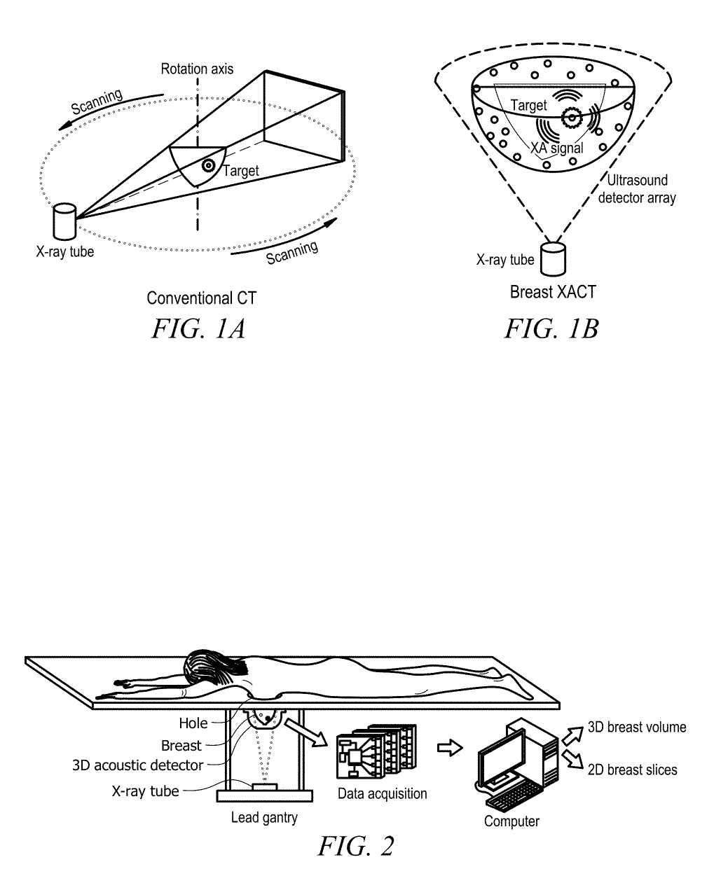

FIG. 1 is a schematic comparing irradiation by conventional breast CT (FIG. 1A) with that of the presently disclosed breast XACT breast imaging system (FIG. 1B). In the presently disclosed XACT breast imaging system, there is no need to rotate the X-ray source and detector to produce a 3D image. Also, there is no need to collimate the X-ray beam or minimize the X-ray detector pixel size, because the spatial resolution is provided by ultrasonic acoustic waves generated by the absorbed X-rays (and detected by transducers), not by scattering of the X-rays themselves. In this way, the XACT breast imaging system can dramatically reduce the radiation dose and provide imaging faster compared to the breast CT, while maintaining high image resolution and image contrast.

In at least one embodiment, an XACT breast imaging system is constructed as shown in FIG. 2. The system includes a cushioned imaging table with an upper surface and a lower surface, and an opening sized to receive a patient's breast. Attached to the lower surface of the table, below the opening, is an acoustic detector cup into which the patient's breast is disposed when the patient is positioned prone facing downwardly on the imaging table. Thus, the system may image the patient's breast individually. The patient can select the most comfortable arm positions for herself. For instance, when the left breast is imaged, the patient can either place her right arm in an arc above her head and left arm along her left side, or stretch both of her arms above her head. Below, various modifications of the table will be disclosed. The construction of the acoustic detector cup is described below in more detail. An X-ray tube is positioned below the table. Alternatively, the X-ray tube is positioned on either side of the table or at any other angle sufficient to direct x-ray pulses towards the patient's breast. For instance, the x-ray tube may be positioned to a side of the breast in order to allow the x-ray pulse to enter the breast, but not substantially enter the chest wall. Compared to other breast CT methods, rotations of the X-ray tube is generally unnecessary because one exposure of an X-ray pulse is generally sufficient to generate a 3D acoustic detection. Therefore, the irradiation duration (i.e., pulse duration) can be reduced to the microsecond level or less (e.g., in a range of 1 ps to about 1 μs) while that of conventional breast CTs usually is more than ten seconds. Additionally, the maximum photon energy of the X-ray tube is adjustable from about 20 kVp to about 100 kVp to adapt to different breast sizes, and the X-ray energy is adjustable, for example to about 75 keV. Outside the X-ray tube, a lead gantry may be used to absorb the scattering X-ray photons and protect the patient from unnecessary radiation dose. A single X-ray pulse emits from the x-ray tube in a straight line towards the patient's breast. The X-ray pulse causes tissue in the breast to expand and contract. The expansion and contraction causes XA waves. As described below, calcifications or other breast abnormalities are denser than normal breast tissue, therefore absorb X-rays more than normal breast tissue, and therefore cause greater expansion and contraction, which causes higher intensity XA waves. Because the X-ray pulse travels in a straight line, there need not be a collimator or other structure connecting the X-ray tube to the acoustic detector cup in order to focus or direct the x-ray pulse.

Acoustic Detector Cup

XA waves are of a spherical nature and propagate in all directions from their point of generation. The spherical XA waves are detected by a hemispherical array of transducers that enables the capture of hundreds of uniformly spaced radial “projections” at a time. The acoustic detector cup is configured to have an upward opening for receiving a breast into an inner space of the detector cup in a natural state and shape, rather than the flattened and compressed configuration that is required in mammography.

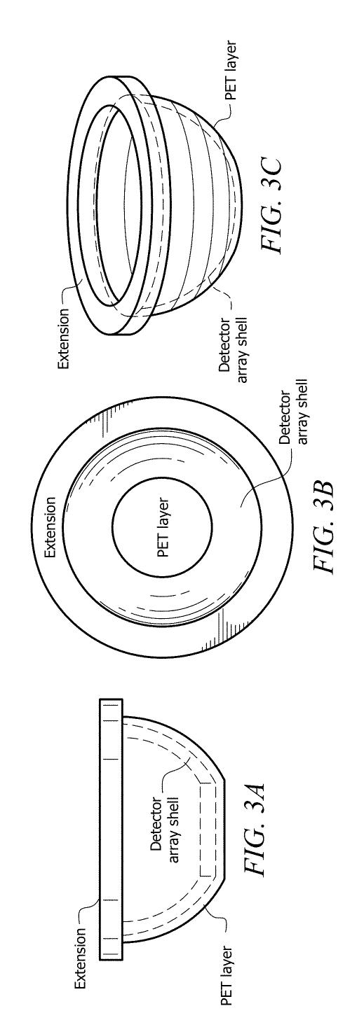

FIGS. 3A-3C show side, top, and perspective views of an acoustic detector cup used in the XACT breast imaging system of the present disclosure. In FIGS. 3A-3C, the cup is constructed with an inner layer and an outer layer. The inner layer is a hemispherically-shaped detector array shell, which may have a polymeric and/or flexible plastic extension for fitting against the lower surface of the table. Embedded within the detector array shell are hundreds (e.g., 100-2,000) of ultrasound transducer elements for receiving XA waves caused by the x-ray pulse. The detector array shell also has a bottom opening through which the X-ray beam can be transmitted, thereby avoiding X-ray energy loss or spectrum deformation before it reaches the breast. In certain embodiments, the plastic extension on the top edge of the shell can be used to fix the acoustic detector cup to the imaging table. In certain embodiments, the outer layer of the acoustic detector cup is a thermoformed shell formed from any suitable material, such as but not limited to, a 1 mm-thick sheet of PET. This outer layer may be optically clear, but must at least be made of a substance through which an X-ray can be transmitted. Before imaging, a quantity of a fluid, such as water or saline, can be placed in the acoustic detector cup to provide an acoustic coupling medium between the ultrasound transducers and the breast. The outer layer helps contain the fluid medium within the inner space of the detector array shell. As noted above, in alternate embodiments, the generated waves can also be detected by a linear transducer array or a 2D transducer rectangular or ring array. In certain non-limiting embodiments, the linear array has 128 transducer elements, the 2D array has 256 (e.g., 4 cm side length) to 1,024 (16 cm side length) transducer elements, and the 3D array has 1,024 transducer elements. The side length of a rectangular detector can vary from 4 cm to 10 cm, for example.

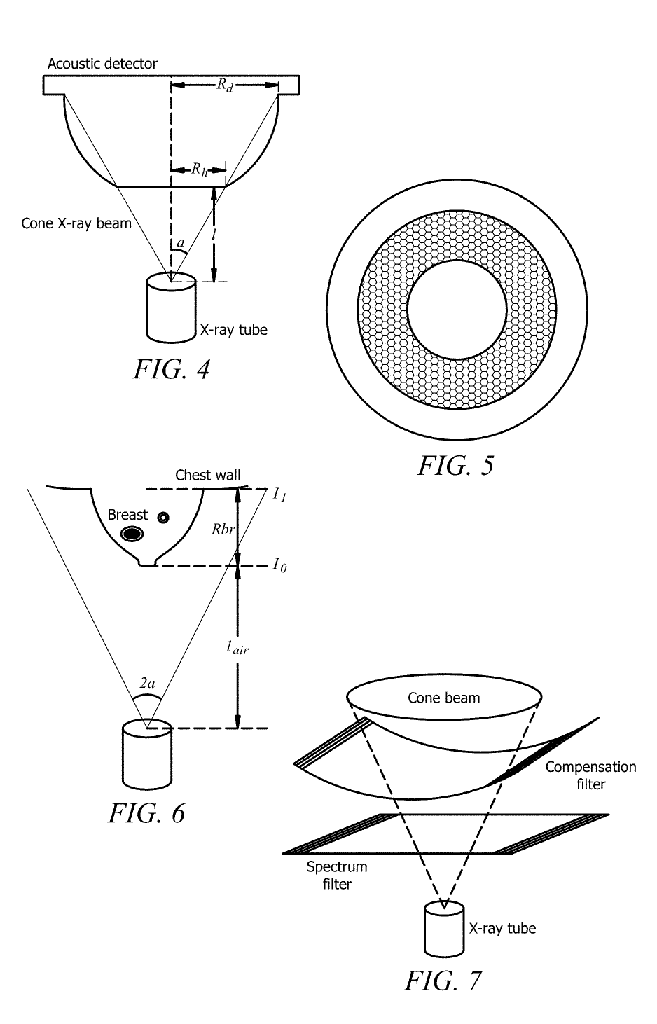

In an embodiment for use with a breast size having a breast radius at chest wall of 8 cm, the radius of the upper opening of the detector array shell Rd will generally exceed 8 cm. As shown in FIG. 4, the bottom opening of the detector array shell, through which the X-ray beam is transmitted, has a radius of Rh. Considering an X-ray tube with a divergence angle of 2α, the mathematical relationship between the X-ray tube location and acoustic detector cup is as follows:

$$\begin{matrix}l = \frac{{R}_{h}}{tan α} , & ( 1 ) \\ {( {R}_{d} / tan α )}^{2} + {R}_{h}^{2} = {R}_{d}^{2} , & ( 2 )\end{matrix}$$

where l is the distance between the X-ray tube and the bottom opening of the detector array shell.

As represented in FIG. 5, tens to hundreds of ultrasound transducer elements are laid out in an array (e.g., in a spiral pattern) on the inner surface of the detector array shell of the acoustic detector cup. In one embodiment, the thread pitch is equivalent to the diameter of a transducer. For example, in one non-limiting embodiment, for a detector array shell having an Rd=10 cm and Rh=5 cm, and transducers each having a 0.5 mm radius, approximately 470 elements are used. Compared with the breast CT, the imaging resolution of which is approximately 200 μm, the XACT breast imaging system of the present disclosure can attain a resolution of 100 μm or less when using, for example, 10 MHz ultrasound transducers having a 70% bandwidth. Even better resolution can be obtained depending on the center frequency of the transducers. In general, the center frequency of each transducer in the array occurs in a range of about 5 MHz to about 12 MHz for imaging different breast sizes.

3D Reconstruction Algorithm

For reconstructing the 3D volume image of the breast, a 3D filtered back-projection algorithm can be used. The acoustic pressure [symbol] ᵣ(t) at detector position r and time t can be expressed as

$${p}_{r} ( t ) = \frac{β {I}_{0} {v}_{s}}{4 π {C}_{p}} τ \frac{d}{dt} ∯ A ( {r}^{′} ) {dr}^{′} , \frac{\vert r - {r}^{′} \vert}{{v}_{s}} = t , \qquad ( 3 )$$

where β denotes the thermal expansion coefficient, C [symbol] is the heat capacity, vₛ is the speed of sound, τ is the radiation pulse width, and I₀ is the intensity. A(r′) is the fractional energy absorption per unit volume of the breast at position r′. Thus, the pressure recorded by detector at position r and time t=|r−r′|/vₛ is the integral (sum) of acoustic pressure waves over the surface of a sphere with a radius |r−r′| in the breast.

Following the pulsed X-ray radiation, time dependent acoustic pressure [symbol] ′_{r,i}(t) is recorded for each transducer i and position r. These signals are recorded by a multi-channel data acquisition system in parallel at a high sampling rate (80 MHz) and high precision (12 bits). These recorded acoustic pressure waves are related to the actual X-ray acoustic pressure signal [symbol] _{r,i}(t) by the equation

zp′ᵢ(t)= [symbol] ᵢ(t)*h(t), (4)

where h(t) is the impulse response of the entire imaging system, including breast tissue and the data receiving circuit, and “*” denotes a convolution operation. Meanwhile, the pressure [symbol] ′_{r,0} (t) is the recorded acoustic pressure due to a point source. Its X-ray acoustic signal is [symbol] _{r,0}(t), [symbol] ′_{r,0}(t)= [symbol] _{r,0}(t)*h(t). As [symbol] ′ᵢ(t) and [symbol] ′₀(t) can be measured from experiments, the X-ray acoustic imaging equation takes the following form:

$$∯ A ( {r}^{′} ) {dr}^{′} = \frac{4 π {C}_{p} {k}_{t}}{β} IFFT ( \frac{{p}_{r , i}^{′} ( ω )}{{p}_{r , 0}^{′} ( ω )} ) , \frac{\vert r - {r}^{′} \vert}{{v}_{s}} = t , \qquad ( 5 )$$

where [symbol] ′_{r,i}(ω) and [symbol] _{r,0}(ω) are the Fourier transforms of [symbol] ′_{r,i}(t) and [symbol] ′_{r,0}(t), respectively. Note that knowledge of the impulse response h(t) of the system is not required. kₜ is a proportionality constant that depends on the illumination geometry and the absorption and scattering property of the breast tissue. The X-ray absorption distribution can then be reconstructed, provided a sufficient number of projections have been acquired. A Fourier filter function of the form

$$F ( ω ) = - {( \frac{ω}{{ω}_{c}} )}^{α} ( 1 + cos ( π ω / {ω}_{c} ) ) , \qquad ( 6 )$$

where 1≤α≤2 and ω_{c} is the cutoff frequency associated with the transducer impulse response, is then used. To reconstruct the 3D breast volume image, the filtered projections can be back-projected to a 3D spherical surface or curved surface according to the detection angle of each ultrasound transducer. Therefore, the 3D reconstruction is accomplished by 1) calculating the projections of the X-ray absorption distribution according to equation (5), 2) filtering the projections using the function (6), 3) back projecting it over a spherical or curved surface, and 4) summing the back projections.

X-Ray Tube

The X-ray tube is a voltage adjustable source. The energy of the tube is set according to the size and density of the patient's breast. The X-ray tube is placed in a specified distance under the detector cup (see equations (1) and (2) above). The X-ray tube's distance from the patient's nipple is variable due to the dimensions of the detector array shell, which depend on the breast size. As illustrated in FIG. 6, the incident X-ray photon energy is I₀, while it is attenuated by breast tissue to I₁ when reaching the chest wall. The percentage of transmission T is obtained as follows:

$$T = {I}_{1} / {I}_{0} = \frac{{l}_{air}^{2}}{{( {l}_{air} + {R}_{br} )}^{2}} exp ( - {μ}_{a} ( e ) {ρ}_{br} {R}_{br} ) , \qquad ( 7 )$$

where lₐᵢᵣ is the distance from the X-ray tube to the patient's nipple, R_{br} is the radius of the patient's breast, and ρ_{br} is the density of the patient's breast. A physician may measure the breast radius R_{br} when the patient is lying on the imaging table with her breast falling naturally through the hole in the table. The breast density ρ_{br} can be approximate to a typical value of 1,020 kg/m³ or estimated by a physician according to the patient's previous medical imaging report. The mass attenuation coefficient μₐ is an energy-dependent coefficient. μₐ decreases in higher X-ray energy, indicating that more photons transmit through the breast and reach the chest wall. To “stop” most of the X-ray photons at the chest wall, T can be set to a small number such as <10%. With the pre-knowledge of lₐᵢᵣ, R_{br} and ρ_{br}, μₐ can be calculated and the optimal X-ray energy can be inferred.

In at least one embodiment of the present disclosure, characteristics of the X-ray beam are mediated by two filters as shown in FIG. 7. A spectrum filter, with selected material and proper material thickness, determines a spectral shape of entrance X-ray photons at a selected kVp. A beam compensation filter is a thickness-variant x-ray beam filter according to the shape of the breast to ensure the x-ray beam penetrates through the breast and stops at or near the chest wall, avoiding unnecessary x-ray exposure to patients. The beam compensation filter produces the proper entrance photon flux distributions determined by the varying breast thickness from the chest wall to the tip of the nipple.

Circuitry and Data Flow

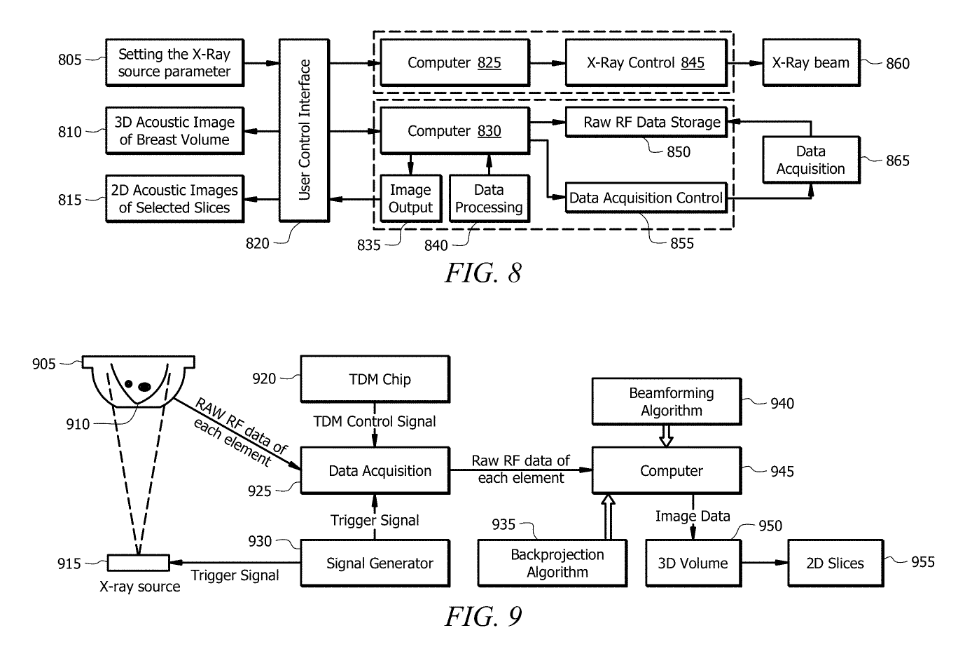

An example of the circuitry of the breast XACT imaging system is shown in FIG. 8. Users control the entire system by a user control interface 820. Using the user control interface 820, a user sets the X-ray source parameter 805. In response, a computer 825 instructs an X-ray control 845, including the exposure voltage, X-ray pulse width, pulse repetition frequency, exposure timing, and total pulse number. As a result, an X-ray source emits an X-ray beam 860. A computer 830, which may be the same as the computer 825, serves the acoustic signal acquisition system. The computer 830 instructs data acquisition control 855. In response, the computer 830 instructs data acquisition 865 of acoustic raw RF data and instructs raw RF data storage 850, for example in a hard disk. Before storing the data, the circuitry may amplify the raw RF data and perform ADC on the amplified data. A data processing module 840 is integrated in the computer 830 to process the recorded acoustic RF data, including acoustic data beamforming and back-projection reconstruction. Finally, the image output 835 is shown via the user control interface 820 as a 3D acoustic image of breast volume 810 and 2D acoustic images of selected slices 815 of the breast on the user control interface 820.

FIG. 9 displays one example of a data flow diagram. A signal generator 930 generates a trigger signal, passes the trigger signal to an X-ray source 915 to instruct the X-ray source 915 to emit an X-ray pulse towards a breast 910 in an acoustic detector cup 905, and passes the trigger signal to instruct acoustic data acquisition 925. In this example, X-ray photon transmission, X-ray energy deposition, and X-ray-acoustic energy conversion are assumed to be completed instantaneously. The acoustic raw RF data of each transducer in the acoustic detector cup 905 is acquired, amplified, and stored in a computer 945, and processed by a delay-and-sum beamforming algorithm 940 and a back-projection algorithm 935 to form a 3D volume 950 and 2D slices 955. The number of acquisition channels is equal to the number of single transducers in the acoustic detector cup. Cables connect the acoustic detector cup to the remaining circuitry. There may be a cable for each acquisition channel and thus each ultrasound transducer element. Alternatively, a TDM chip 920 is used to control the data acquisition sequencing.

Alternatives of the Imaging System

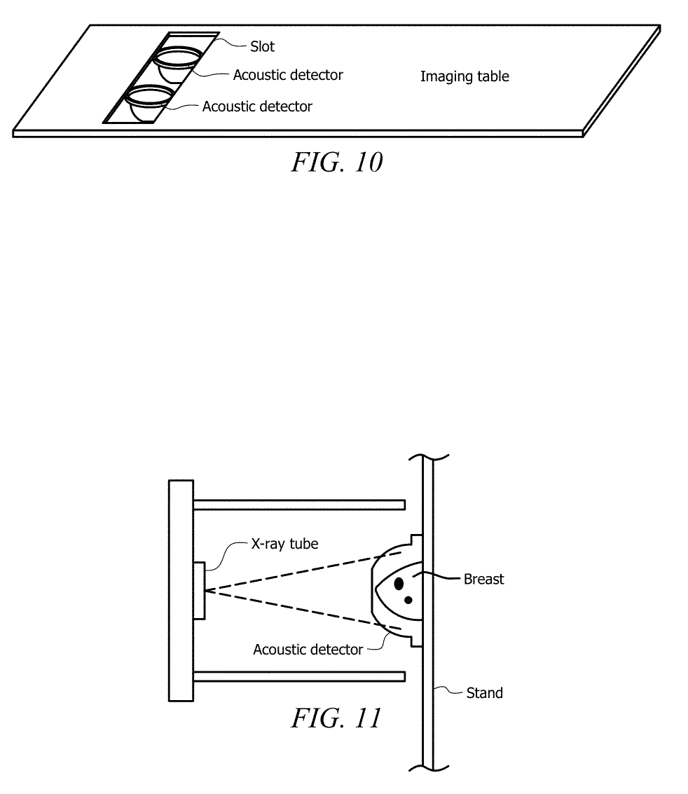

In an alternative embodiment, the imaging table can be replaced with a table shown in FIG. 10. This table is similar to the table of FIG. 2, except that a pair of openings and a slot are provided. One or two acoustic detector cups are mounted to the slot under the table. The acoustic detector(s) can be positioned at various locations along the slot to adjust to an individual patient. If two detectors are used, the distance between them is adjusted according to the geometric relationship between the patient's breasts. Each detector is equipped with an X-ray tube. Alternatively, the imaging can be performed while the patient is standing. As shown in FIG. 11, in such an imaging system, one or a pair of acoustic detector cups are supported by a stand to support one breast or two breasts of a standing patient. When two acoustic detector cups are used, the distance between the two cups is adjusted according to the locations of the patient's breasts.

Sensitivity of XACT Breast Imaging System

The breast XACT is sensitive to the tissue characteristic variations. In breast XACT, acoustic signals generated by X-rays are detected. When thermal confinement is satisfied, the following XA equation for an arbitrary absorbing target with an arbitrary excitation source is obtained:

$$( {∇}^{2} - \frac{1}{{v}_{s}^{2}} \frac{{\partial }^{2}}{\partial {t}^{2}} ) p ( \vec{r} , t ) = - \frac{β}{{C}_{p}} \frac{\partial H ( \vec{r} , t )}{\partial t} , \qquad ( 8 )$$

where p(\vec{r},t) source denotes the acoustic pressure rise at location \vec{r} and time t, vₛ is the speed of sound, β denotes the thermal coefficient of volume, Cₚ denotes the specific heat capacity at a constant pressure, and H(\vec{r},t) is the heating function. The left-hand side of equation (8) describes wave propagation in an inviscid medium, whereas the right-hand side represents the source. Equation (8) shows that the propagation of an X-ray induced acoustic pressure wave is driven by the first time derivative of the heating function H(\vec{r},t). Therefore, time-invariant heating does not generate an XA pressure wave; only time-variant heating does. For such a short X-ray pulse, the fractional volume expansion is negligible and the local pressure rise p₀ immediately after the X-ray excitation can be written as p₀=ΓηₜₕμF, where Γ is a Grueneisen parameter, ηₜₕ is the percentage of absorbed energy that is converted to heat, μ denotes the X-ray absorption coefficient, and F denotes the X-ray fluence in joules per centimeter square. Hence, the X-ray-induced signal (p₀) is proportional to the X-ray absorption coefficient μ. Focusing on the variation in the local absorption coefficient, Δp₀/p₀=Δμ/μ, where Δ indicates a small variation in the modified variable. Here, the effect of Δμ on Δp₀ through F is neglected. Considering that μ=σρN_{A}/A, where ρ is the mass density, σ is the absorption cross section, N_{A} is the Avogadro number, and A is the atomic number, then the acoustic pressure variation Δp₀ is proportional to the variation of tissue characteristics ΔσΔρ. Δρ denotes the change in tissue density, while Δσ reflects the change in tissue compositions. Therefore, any fractional change in tissue characteristic translates into an equal amount of fractional change in the X-ray induced signal.

Meanwhile, due to Δp₀/p₀=Δμ/μ, XACT breast imaging is only sensitive to X-ray absorption (i.e., a given percentage change in the X-ray absorption coefficient yields the same percentage change in the X-ray acoustic amplitude), and not to X-ray scattering. It naturally filters out the scattering X-rays. There can be less background and increased signal to noise ratio, and therefore increased sensitivity to X-ray absorption compared with conventional X-ray imaging. The ultimate detection sensitivity is limited mainly by thermal noise.

Radiation Dose of XACT Breast Imaging System

As noted above, use of the presently disclosed XACT breast imaging reduces radiation dose. An NEP model is used for calculating the minimal radiation dose in XACT breast imaging. In XACT breast imaging, noise mainly arises from three sources: thermal acoustic noise from the medium, thermal noise from the ultrasonic transducer, and electronic noise from the amplifier. NEP can be expressed as a spectral density with units of Pa/\sqrt{Hz} as follows:

$$NEP ( f ) = \sqrt{{k}_{B} T [ 1 + \frac{{F}_{n}}{η ( f )} ] {Z}_{a} / A} , \qquad ( 9 )$$

where k_{B} is the Boltzmann constant (1.38×10−23 J/K), T is the absolute temperature of the medium in Kelvin, and Fₙ denotes the noise factor of the amplifier and has a typical value of 2 over its bandwidth. For an ultrasound transducer with a center frequency of f₀ and a detection bandwidth of Δf, it can be assumed that the detector efficiency is uniform such that η(f)≈η(f₀), and η(f₀) have a value of 0.5 (−3 dB). Zₐ denotes the characteristic acoustic impedance of the medium (1.5×10⁶ Rayls for water), and A is the size of the detector.

The minimal radiation dose to generate a detectable acoustic signal is calculated as follows:

$$Dose = \frac{NEP ( f ) \sqrt{BW} {C}_{p}}{β {v}_{s}^{2} ρ} , \qquad ( 10 )$$

where \sqrt{BW} is a bandwidth of the acoustic detector, Cₚ is the heat capacity, β denotes a thermal expansion coefficient of an absorption target, υₛ is a speed of ultrasound in breast tissue, and ρ is a density of the absorption target. If the breast XACT is used for detecting breast calcifications with less than a 100 μm resolution, when a 0.1 ns pulsed X-ray is used as the excitation source, the minimal X-ray dose for generating a detectable acoustic signal is 1.1 mGy, which is approximately 10 times less radiation dose than the newly-FDA-approved breast CT, which requires an X-ray dose of about 16 mGy. Table I compares XACT breast imaging as presently disclosed to representative breast CTs.

TABLE 1
XACT breast imaging vs. several representative breast CT technologies

| | | UC Davis | Koning Standard | Duke/ |
|---|---|---|---|---|
| Parameter | XACT | (Doheny) | (UMass†) | Zumatek |
| X-ray pulsing | Pulse (60 ns) | Pulsed (3~8 ms) | Pulsed (8 ms) | Pulsed (25 ms) |
| No. of projections | 1 | 500~800 | 300 | 300 |
| Imaging resolution | <100 μm | <300 μm | <270 μm | <200 μm |
| Imaging speed | <0.04 s | 16.6 s | 10 s | 1.5 min |
| Dose | <1.1 mGy | 5.4 mGy | 16 mGy | 4.5 mGy |
| Detector type | Ultrasound | CMOS + CsI:Tl | a-Si + CsI:Tl | a-Si + CsI:Tl |

The following describes a minimal dose needed for calcification detection. Acoustic detector sensitivity is quantified by the noise equivalent pressure (NEP), where

$$NEP = \sqrt{\frac{{k}_{B} T [ 1 + \frac{{F}_{n}}{η ( f )} ] {Z}_{a}}{A}} * \sqrt{BW} , \qquad ( 11 )$$

where Fₙ has a typical value of 2 over its bandwidth. For an ultrasound transducer with a center frequency of f₀ and a detection bandwidth Δf, it may be assumed that the detector efficiency is uniform such that η(f)≈η(f₀), and η(f₀) has a value of 0.5 (−3 dB). k_{B} is the Boltzmann constant (1.38×10⁻²³ J/K), and T is the absolute temperature of the medium in Kelvin (300 K). Zₐ denotes the characteristic acoustic impedance of the medium (1.5×10⁶ Rayls/m² for water). A is the surface area of a single detector element with radium r=7 mm).

Ultrasound detectors are distributed in a hemisphere with R=80 mm. A total number of the detectors is

$${N}_{ust} = \frac{2 π {R}^{2}}{π {r}^{2}} . \qquad ( 12 )$$

After back-projection, the summed total noise caused by Nᵤₛₜ ultrasound detectors is

[symbol]

ₙₒᵢₛₑ=\sqrt{Nᵤₛₜ}·NEP. (13)

To ensure the SNR is above 4, then the minimal required pressure caused by a calcification is

[symbol]

₁=4NEP\sqrt{Nᵤₛₜ}. (14)

On the other hand, the initial pressure rise for a calcification is [symbol] ₀, where

$${p}_{0} = \frac{β {v}_{s}^{2}}{{C}_{p}} {μ}_{{a}_{ca}} {ρ}_{ca} F . \qquad ( 15 )$$

After penetrating 8 cm of breast tissue (attenuation is 0.75 dB for breast tissue), the acoustic pressure that reaches any detector element is

$$\begin{matrix}{p}_{receive} = \frac{β {v}_{s}^{2}}{{C}_{p}} {μ}_{{a}_{ca}} {ρ}_{ca} F \cdot atten \cdot \frac{1}{{N}_{ust}} & ( 16 ) \\ atten = 10 \frac{\frac{\frac{0.75 dB}{cm}}{MHz} * 5.5 MHz * 8 cm}{20} = 0.022 . & ( 17 )\end{matrix}$$

After back-projection, the total pressure is Nᵤₛₜ times of [symbol] _{receive}, where

$${p}_{2} = \frac{β {v}_{s}^{2}}{{C}_{p}} {μ}_{{a}_{ca}} {ρ}_{ca} F \cdot atten . \qquad ( 18 )$$

If [symbol] ᵢ= [symbol] ₂, then the incident x-ray fluence Fᵢₙ can be determined as follows:

$${F}_{in} = 4 NEP \sqrt{{N}_{ust}} \cdot \frac{{C}_{p}}{β {v}_{s}^{2} {μ}_{{a}_{ca}} {ρ}_{ca} F \cdot atten} . \qquad ( 19 )$$

After penetrating a distance dz in small voxel (dx, dy, dz), the exit fluence is

Fₒᵤₜ=Fᵢₙ·e^{-μ}^{_{abr}}^{ρ}^{_{br}}^{·dz}. (20)

The energy deposited inside the voxel is

E_{depo}=(Fᵢₙ−Fₒᵤₜ)·Area=(Fᵢₙ−Fₒᵤₜ)·dxdy. (21)

Then, the dose can be calculated by

$$dose = \frac{{E}_{depo}}{Mass} = \frac{{E}_{depo}}{dxdydz \cdot {ρ}_{br}} . \qquad ( 22 )$$

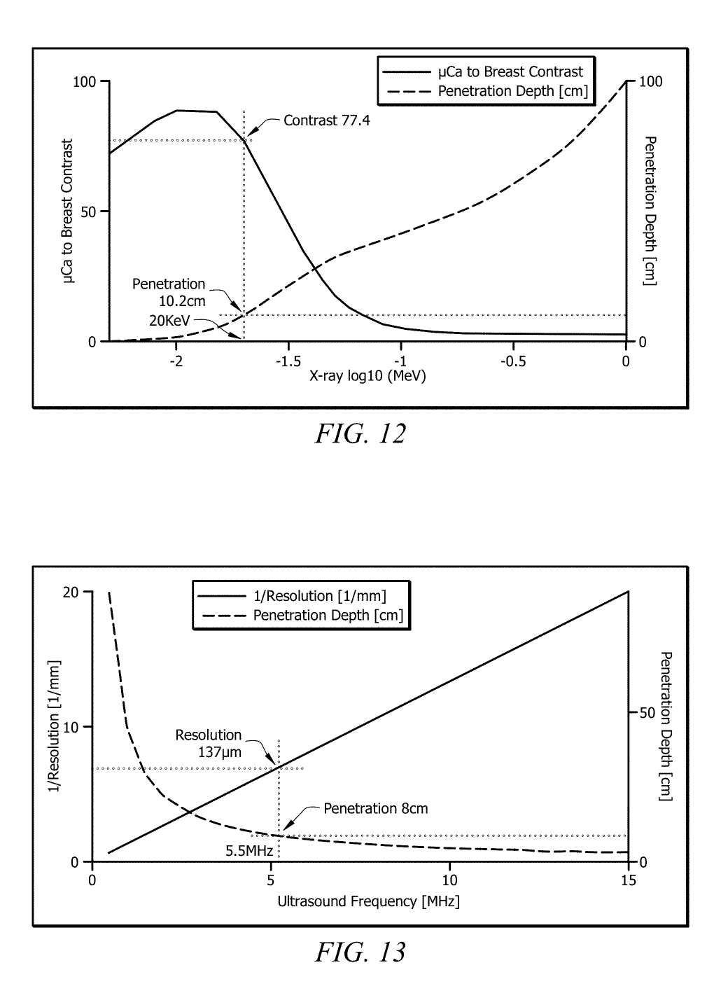

FIG. 12 is a graph illustrating x-ray energy dependent x-ray penetration depth versus calcification-breast tissue imaging contrast. FIG. 12 shows that there is a tradeoff between breast contrast and penetration depth. Based on the intersection of the areas underneath the curves, an x-ray energy of between 20 keV and 100 keV is desired.

FIG. 13 is a graph illustrating frequency-dependent ultrasound penetration depth versus spatial resolution. FIG. 13 shows that there is a tradeoff between resolution and penetration depth. Based on the intersection of the areas underneath the curves, a frequency of between 5.5 MHz and 15 MHz is desired.

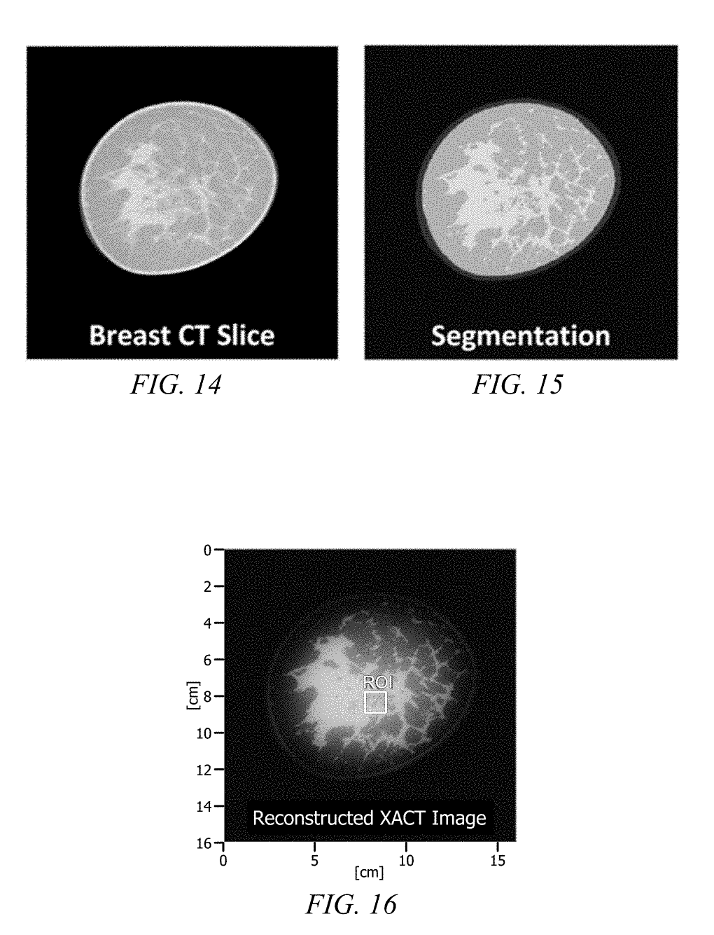

FIG. 14 is an image of an observed conventional breast CT. FIG. 15 is a segmentation of the observed breast CT in FIG. 14. Segmentations are helpful because they show different layers, and thus different types of tissue, in the breast.

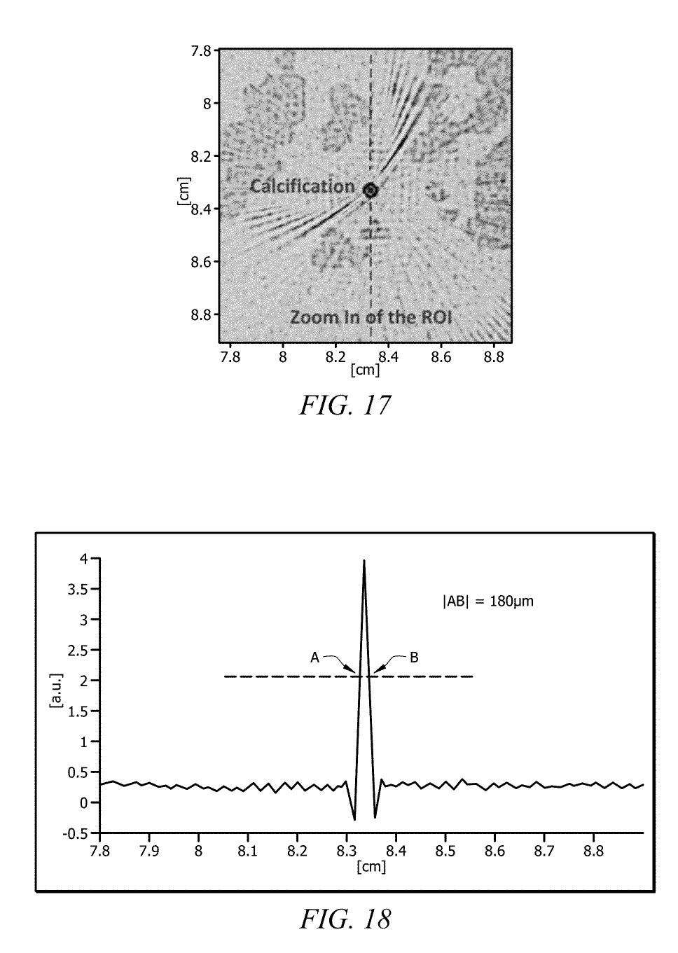

FIG. 16 is a simulated XACT image based on the observed conventional breast CT slice in FIG. 14. The XACT image comprises an ROI. FIG. 17 is an image of the ROI in FIG. 16. As can be seen, there is a dot in the middle of the ROI. The dot indicates a stronger signal intensity and thus a possible calcification. FIG. 18 shows the signal intensity for the image of the ROI in FIG. 17 across the dotted line. As can be seen, there is an intensity spike between 8.3 cm and 8.4 cm corresponding to the dot and possible calcification in FIG. 17.

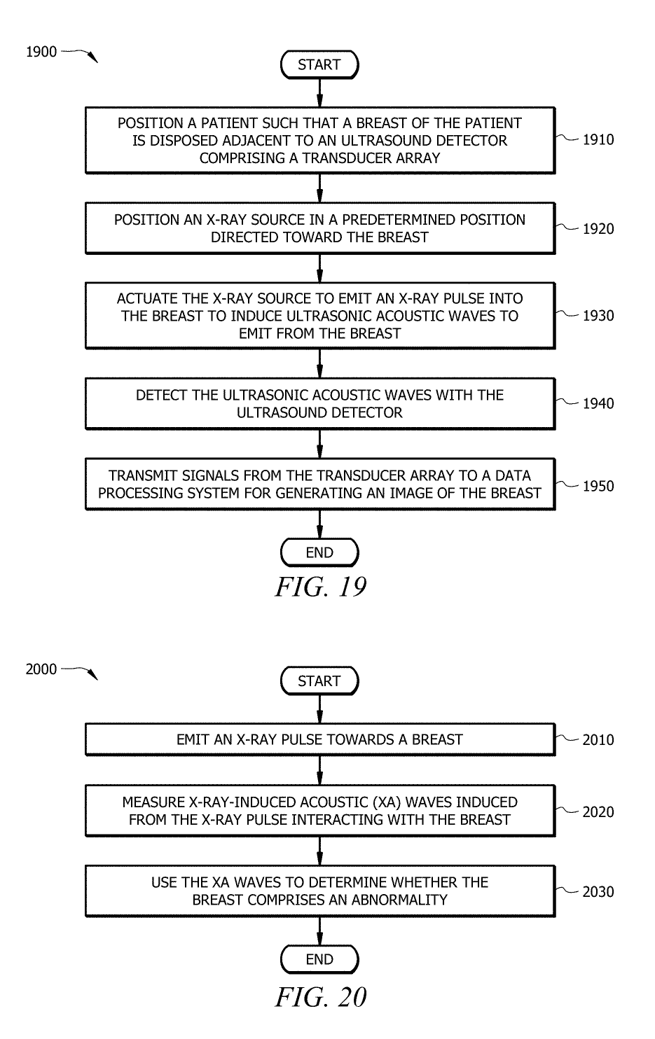

FIG. 19 is a flowchart illustrating a method 1900 of breast imaging according to an embodiment of the disclosure. At step 1910, a patient is positioned such that a breast of the patient is disposed adjacent to an ultrasound detector comprising a transducer array. The transducer array comprises a plurality of ultrasonic transducer elements. At step 1920, an X-ray source is positioned in a predetermined position directed toward the breast. At step 1930, the X-ray source is actuated to emit an X-ray pulse into the breast to induce ultrasonic acoustic waves to emit from the breast. The X-ray pulse has a duration in a range of 1 ps to 1 μs. At step 1940, the ultrasonic acoustic waves are detected with the ultrasound detector. Finally, at step 1950, signals from the transducer array are transmitted to a data processing system for generating an image of the breast.

FIG. 20 is a flowchart illustrating a method 2000 of breast imaging according to another embodiment of the disclosure. At step 2010, an X-ray pulse is emitted towards a breast. At step 2020, XA waves induced from the x-ray pulse interacting with the breast are measured. Finally, at step 2030, the XA waves are used to determine whether the breast comprises an abnormality.

In at least one embodiment, the present disclosure is directed to an X-ray computed tomography breast imaging method comprising positioning a patient such that a breast of the patient is disposed adjacent to an ultrasound detector comprising a transducer array, wherein the transducer array comprises a plurality of ultrasonic transducer elements; positioning an X-ray source in a predetermined position directed toward the breast; actuating the X-ray source to emit an X-ray pulse into the breast to induce ultrasonic acoustic waves to emit from the breast, wherein the X-ray pulse has a duration in a range of 1 ps to 1 μs; detecting the ultrasonic acoustic waves with the ultrasound detector; and transmitting signals from the transducer array to a data processing system for generating an image of the breast. In some embodiments, the ultrasound detector is a 3D hemispherical cup comprising an inner space and an inner layer, and wherein the inner layer comprises the transducer array; the method further comprises at least partially disposing the breast within the inner space; the method further comprises further positioning the patient such that the breast is not compressed.

In another embodiment, the present disclosure is directed to an acoustic detector cup comprising: a breast opening configured to receive a breast; an outer layer coupled to the breast opening and configured to allow an X-ray pulse to pass through; and an inner layer coupled to the outer layer and comprising a detector array shell, wherein the detector array shell comprises a plurality of ultrasound transducer elements configured to receive XA waves caused by the X-ray pulse interacting with the breast. In some embodiments, the breast opening is further configured to further receive the breast in a natural shape without compression; the outer layer is further configured to receive and contain a quantity of fluid in order to provide an acoustic coupling medium between the breast and the ultrasound transducer elements; the outer layer is a thermoformed shell; the outer layer comprises a material suitable for allowing the X-ray pulse to pass through; the material comprises PET; the outer layer is optically clear; the inner layer comprises an extension configured to attach to a mounting surface; the extension is polymeric or plastic; the detector array shell is hemispherical; the detector array shell comprises an X-ray opening configured to receive the X-ray pulse; the ultrasound transducer elements are further configured to convert the XA waves into electrical signals; the detector array shell is configured to: couple to a plurality of cables corresponding to the ultrasound transducer elements; and pass the electrical signals from the ultrasound transducer elements to the cables. In some embodiments, an X-ray computed tomography breast imaging system comprises the acoustic detector cup described above.

In yet another embodiment, the present disclosure is directed to a method comprising: emitting an X-ray pulse towards a breast; measuring XA waves induced from the X-ray pulse interacting with the breast; and using the XA waves to determine whether the breast comprises an abnormality. In some embodiments, the method further comprises producing a 3D image of the breast using the X-ray pulse without additional X-ray pulses; the X-ray pulse comprises a duration of less than 1 μs.

What is claimed is:

1. An X-ray computed tomography breast imaging method comprising:
positioning a patient such that a breast of the patient is disposed adjacent to an ultrasound detector comprising a transducer array, wherein the transducer array comprises a plurality of ultrasonic transducer elements;
positioning an X-ray source in a predetermined position directed toward the breast;
actuating the X-ray source to emit an X-ray pulse into the breast to induce ultrasonic acoustic waves to emit from the breast, wherein the X-ray pulse has a duration in a range of 1 picosecond (ps) to 1 microsecond (μs);
detecting the ultrasonic acoustic waves with the ultrasound detector; and
transmitting signals from the transducer array to a data processing system for generating an image of the breast.

2. The method of claim 1, wherein the ultrasound detector is a three-dimensional (3D) hemispherical cup comprising an inner space and an inner layer, and wherein the inner layer comprises the transducer array.

3. The method of claim 2, further comprising at least partially disposing the breast within the inner space.

4. The method of claim 1, further comprising further positioning the patient such that the breast is not compressed.

5. An acoustic detector cup comprising:
a breast opening configured to receive a breast;
an outer layer coupled to the breast opening and configured to allow an X-ray pulse to pass through; and
an inner layer coupled to the outer layer and comprising a detector array shell,
wherein the detector array shell comprises a plurality of ultrasound transducer elements configured to receive X-ray-induced acoustic (XA) waves caused by the X-ray pulse interacting with the breast.

6. The acoustic detector cup of claim 5, wherein the breast opening is further configured to further receive the breast in a natural shape without compression.

7. The acoustic detector cup of claim 5, wherein the outer layer is further configured to receive and contain a quantity of fluid in order to provide an acoustic coupling medium between the breast and the ultrasound transducer elements.

8. The acoustic detector cup of claim 5, wherein the outer layer is a thermoformed shell.

9. The acoustic detector coup of claim 5, wherein the outer layer comprises a material suitable for allowing the X-ray pulse to pass through.

10. The acoustic detector cup of claim 9, wherein the material comprises polyethylene terephthalate (PET).

11. The acoustic detector cup of claim 5, wherein the outer layer is optically clear.

12. The acoustic detector cup of claim 5, wherein the inner layer comprises an extension configured to attach to a mounting surface.

13. The acoustic detector cup of claim 12, wherein the extension is polymeric or plastic.

14. The acoustic detector cup of claim 5, wherein the detector array shell is hemispherical.

15. The acoustic detector cup of claim 5, wherein the detector array shell comprises an X-ray opening configured to receive the X-ray pulse.

16. The acoustic detector cup of claim 5, wherein the ultrasound transducer elements are further configured to convert the XA waves into electrical signals.

17. The acoustic detector cup of claim 16, wherein the detector array shell is configured to:
couple to a plurality of cables corresponding to the ultrasound transducer elements; and pass the electrical signals from the ultrasound transducer elements to the cables.

18. An X-ray computed tomography breast imaging system comprising the acoustic detector cup of claim 5.

19. A method comprising:
emitting an X-ray pulse towards a breast;
measuring X-ray-induced acoustic (XA) waves induced from the X-ray pulse interacting with the breast; and
using the XA waves to determine whether the breast comprises an abnormality.

20. The method of claim 19, further comprising producing a three-dimensional (3D) image of the breast using the X-ray pulse without additional X-ray pulses.

21. The method of claim 19, wherein the X-ray pulse comprises a duration of less than 1 microsecond (μs).
